(19) United States
(12) Patent Application Publication
(10) Pub. No.: US 2024/0230668 A1
(43) Pub. Date: Jul. 11, 2024

(54) PRETREATMENT METHOD FOR QUANTITATIVE DETECTION OF UNDENATURED TYPE II COLLAGEN IN COLLAGEN PRODUCT OR CARTILAGE, AND APPLICATION

(71) Applicant: Beijing SEMNL BIOTECHNOLOGY CO., LTD., Anhui (CN)

(72) Inventors: Lige LIU, Beijing (CN); Zheng YAN, Beijing (CN); Aiqing LIU, Beijing (CN); Haiyan WANG, Beijing (CN); Guoqing ZOU, Beijing (CN)

(21) Appl. No.: 18/557,508

(22) PCT Filed: Apr. 26, 2021
(86) PCT No.: PCT/CN2021/089978

§ 371 Date: Oct. 26, 2023

(51) Int. Cl. G01N 33/68; G01N 33/12

(52) U.S. Cl. CPC G01N 33/6887; G01N 33/12; G01N 2333/78; G01N 2333/96477; G01N 2333/966

(57) ABSTRACT

A pretreatment method for detection of an undenatured type II collagen in a cartilage collagen product or a cartilage is proposed. The method can separate an undenatured type II collagen from a complex environment system including proteoglycan, a hydrolyzed collagen, open-chain denatured collagen and the like, and present a dissolved state, thereby facilitating subsequent further qualitative and quantitative detection.

### FIELD

The present invention relates to a pre-treatment method for collagen assay, and specifically to a pre-treatment method for the quantitative assay of non-denatured type II collagen in a chicken cartilage and its product.

### BACKGROUND

Collagen is one of the most abundant proteins and is widely present in humans and animals. It is the main component of the extracellular matrix and plays an important role in living organism. Type I collagen is one of the biomaterials early approved by the FDA and SFDA due to its low immunogenicity, good biocompatibility, and biodegradability. The active collagen that maintains the natural triple helix conformation can be self-assembled or covalently combined with fibronectin, macromolecular polysaccharide to produce biomaterials with different structures and functions. In recent years, several studies have shown that non-denatured type II collagen which maintains its natural triple helix conformation has an important role in joint health through oral immune tolerance. 250-270 peptide segment of non-denatured type II collagen is recognized by T cells in the organism, which establishes an immune response mechanism, and after being absorbed into circular system through M cells in arch area of Pai's spot in intestinal mucosa, stimulates the immune system again to produce an immune factor. The immune factor gradually stimulates the immune tolerance to the patient site, which stops the erosion and destruction of the cartilage tissue, and then repairs the damaged cartilage. However, the hydrolyzed and open-chain type II collagen does not have this immune effect. Therefore, oral administration of a certain dose of non-denatured type II collagen may produce immune tolerance in the body and thus control the symptoms of arthritis.

There are many methods to detect collagen content and type, but there is no commonly accepted method for the detection of non-denatured type II collagen that maintains its natural conformation. The commonly used methods for the detection of type II collagen in tissues or tissue fluids are histochemical staining, immunological assays, ELISA determination of telopeptides, hydroxyproline (HYP) colorimetric assay, etc. These histochemical methods are expensive in terms of reagents and testing instruments. Moreover, for type II collagen products and end products with type II collagen added after refinement and purification, the tissue structure has been destroyed and is no longer available for tissue testing, and it is difficult to identify collagen denatured during processing. The ELISA methods have unparalleled advantages in the identification of collagen types, as it requires the separation of non-denatured collagen from denatured or hydrolyzed collagen that does not have a triple helix structure, either by using the antigenic site of the telopeptide or triple helix or by hydroxyproline colorimetry. ELISA uses specific recognition between antigen and antibody to characterize, type, and quantify collagen based on difference in antigenic determinant of different types of collagens, and requires the target analyte to be soluble.

Type II collagen is mainly found in cartilage and is insoluble in water like other collagens. Commonly used methods of collagen extraction include acid method and pepsin method. For example, soluble collagen can be obtained by acid swelling of type I collagen for a certain period etc. (Pieper J S et al., biomaterials 1999, 20: 847-858), but the extraction level is still low. The efficiency of the low-temperature pepsin method is low in a short time and the obtained collagen is still aggregated, while a longer time is needed to improve the efficiency of enzymatic digestion, during which the free monomeric collagen may be denatured and hydrolyzed by pepsin, which makes the quantification by ELISA difficult.

Unlike type I collagen, the cartilage used to prepare type II collagen has more proteoglycans in addition to collagen. Even after prolonging the duration of acid treatment and increasing the number of acid treatments, the cartilage polysaccharide still binds to type II collagen, and the acid swelling of natural undenatured type II collagen is very unsatisfactory. Even after multiple treatments, it remains aggregated and fibrous, with antigenic determinants wrapped inside the fibers, which renders extraction and detection difficult.

### SUMMARY

In view of the above deficiencies in the prior art, the present invention provides a pre-treatment method for the detection of non-denatured type II collagen in a collagen product or cartilage raw material, which is capable of separating non-denatured collagen from a complex environmental system containing proteoglycans, hydrolyzed collagen, open-chain denatured collagen, etc., and presenting it in a soluble state for further qualitative and quantitative detection subsequently.

A pre-treatment method for the detection of non-denatured type II collagen in a collagen product or cartilage raw material as described in the present invention comprises the steps of:
(1) washing a sample of the collagen product or raw material to be tested with a buffer containing a neutral salt or guanidine hydrochloride for at least 16 h;
(2) optionally, if a buffer containing a neutral salt is used for washing in step (1), washing with a buffer containing guanidine hydrochloride for at least 16 h may be performed after step (1);
(3) washing with water for 1 to 3 times;
(4) ultrasonic swelling with 0.05 mol/L acetic acid (HAc), temperature ≤25° C.;
(5) pepsin digestion for at least 16 h, temperature ≤37° C.;
(6) elastase digestion for at least 10 h, temperature ≤10° C.;

The pre-treatment method for cartilage sample also includes pre-treatment steps such as removal of selected fleshy fascia and the like, degreasing, and crushing. Among them, degreasing is preferably performed by 1 part of cartilage to 9 parts of cold water, using 20 parts of chloroform-methanol-water (1:2:0.8) solution. Optionally, the sample to be tested is crushed to obtain a powder no larger than 100 mesh. The method of obtaining a sample of the cartilage collagen product optionally comprises grinding the product to 100 mesh-200 mesh or not greater than 100 mesh.

In some embodiments, the neutral salt in step (1) is optionally MgCl₂, NaCl, preferably MgCl₂. In some embodiments, the buffer in steps (1) or (2) is optionally PBS buffer or Tris-HCl buffer, preferably Tris-HCl buffer. In some embodiments, the Tris-HCl buffer has a concentration of 50-100 mmol/L, a pH of 7.2-7.5, and contains 25 mmol/L EDTA-Na₂ and 2 mmol/L N-Ethylmaleimide (NEM). In some embodiments, the concentration of MgCl₂ is between 1-6 mol/L, preferably 2-6 mol/L, more preferably 3-6 mol/L, more preferably 3-5 mol/L, more preferably 3-4 mol/L. In some embodiments, the concentration of guanidine hydrochloride is 1-4 mol/L, preferably 3-4 mol/L.

In some embodiments, steps (1)-(3) are performed at 4-10° C.

In some embodiments, the number of sonication in step (4) is 1 to 2, each not exceeding 15 min, the temperature during sonication is controlled to no higher than 25° C., and the power of sonication is optionally 100-500 W.

In some embodiments, the concentration of the pepsin solution described in step (5) is 0.1-5 mg/mL, preferably 0.5-4.5 mg/mL, more preferably 0.5-4 mg/mL, more preferably 0.5-3.5 mg/mL, more preferably 0.5-3 mg/mL, more preferably 0.5-2.5 mg/mL, more preferably 0.5-2 mg/mL, more preferably 0.5-1.5 mg/mL, more preferably 0.8-1.5 mg/mL, more preferably 0.8-1.2 mg/mL.

In some embodiments, the duration of the enzymatic digestion described in step (5) is 16-72 h, preferably 16-65 h, more preferably 16-60 h, more preferably 16-55 h, more preferably 16-50 h, more preferably 16-45 h, more preferably 16-45 h, more preferably 16-40 h, more preferably 16-35 h, more preferably 16-30 h, more preferably 16-25 h, more preferably 16-22 h, more preferably 16-20 h; the temperature of the enzymatic digestion is optionally 4° C.-37° C., optionally ≤30° C., optionally ≤25° C., optionally ≤20° C., preferably 6° C.-30° C., more preferably 8° C.-30° C., more preferably 10° C.-30° C., more preferably 15° C.-30° C., more preferably 18° C.-30° C., more preferably 20° C.-30° C., more preferably 25° C.-30° C.

In some embodiments, the final concentration of elastase in solution as described in step (6) is 0.05-0.3 mg/mL, preferably 0.05-0.25 mg/mL, more preferably 0.05-0.2 mg/mL, more preferably 0.05-0.15 mg/mL, more preferably 0.08-0.12 mg/mL, more preferably 0.1 mg/mL.

In some embodiments, the duration of the enzymatic digestion described in step (6) is 10-48 h, preferably 10-40 h, more preferably 10-35 h, more preferably 10-30 h, more preferably 15-30 h, more preferably 15-25 h, more preferably 16-25 h, more preferably 16-22 h, more preferably 16-20 h, more preferably 16-18 h, more preferably 16 h; the temperature of the enzymatic digestion is 2-10° C., more preferably 2-8° C., more preferably 2-6° C., more preferably 3-6° C., more preferably 4-6° C.

In some embodiments, the acetic acid sonication swelling of step (4) may be repeated, preferably 1-2 times; after each sonication swelling, centrifugation is performed and the supernatant is collected, and if the sonication step is repeated, the supernatant is combined to obtain a combined solution.

In some embodiments, step (5) further comprises adjusting the pH of the supernatant or combined solution obtained after sonication in step (4) to ≤3.0, preferably 2.0-3.0, more preferably 2.5-3.0, more preferably 2.5-2.8, using formic acid.

In some embodiments, step (6) further comprises adding 10-fold TBS buffer to the enzymatic digest solution obtained in step (5) and adjusting the pH to 7.5-9.0, preferably 7.5-8.5, more preferably 8.0-8.5, more preferably 8.0-8.1 with 2 mol/L NaOH. The 10-fold TBS buffer is a buffer containing 0.4 mol/L NaCl and 10 mmol/L CaCl₂) in a 1 mol/L Tris solution.

In some embodiments, step (6) further comprises centrifugation after the enzymatic digestion and collecting the supernatant.

In some embodiments, the above method further comprises diluting the supernatant obtained in step (6) with 0.05 mol/L PBS buffer (pH 7.4). A person skilled in the art is able to determine a suitable dilution fold depending on the assay or kit being used.

In another aspect, the above method can also be used to extract non-denatured type II collagen from cartilage raw material. Specifically, the supernatant obtained after steps (1)-(6) above may be further purified by a person skilled in the art, e.g., by salinization and dialysis, to remove the hydrolyzed and/or open-chain collagen that may have been generated during the extraction process. For example, the supernatant was salted with NaCl at a final concentration of 2 mol/L as described above, and the precipitate was dialyzed with 0.05 mol/L PBS (pH 7.4) for 24 h, with dialysate changed every 4 h. The experimental results showed that only a very small amount of non-denatured type II collagen was lost by the operation of salinization and dialysis.

In another aspect, the present invention provides a kit for the detection of non-denatured type II collagen in a collagen product or cartilage raw material or for the pretreatment of the assay. The kit comprises a buffer solution containing a neutral salt or guanidine hydrochloride, a pepsin solution and an elastase solution.

In some embodiments, the concentration of guanidine hydrochloride in the buffer solution containing guanidine hydrochloride in the kit is between 1-6 mol/L, preferably 3-4 mol/L. In some embodiments, the buffer solution containing a neutral salt or guanidine hydrochloride is prepared by a method comprising the steps of dissolving the neutral salt or guanidine hydrochloride in 0.1 mol/L Tris-HCl buffer and stirring well.

In some embodiments, the concentration of pepsin in the pepsin solution in the kit is optionally >0.1 mg/mL, optionally 0.1-5 mg/mL, optionally 0.5-1.5 mg/mL, preferably >0.5 mg/mL, more preferably >1 mg/mL, more preferably 1-20 mg/mL. In some embodiments, the concentration of elastase in the elastase solution in the kit is optionally >0.01 mg/mL, optionally 0.01-2 mg/mL, preferably >0.05 mg/mL, more preferably >0.1 mg/mL, more preferably 0.1-2 mg/mL.

“Pepsin” is a digestive protease secreted by the gastric chief cell in the stomach and functions to break down proteins in food into small peptide fragments. Pepsin can be obtained by a variety of known methods or routes by those skilled in the art. In some embodiments, the pepsin used in the present invention is porcine pepsin (from porcine gastric mucosa), for example, P6887 (Sigma-Aldrich).

“Elastase” is a serine protease that is widely found in the mammalian pancreas. Elastase can be obtained by a variety of known methods or routes by those skilled in the art. In some embodiments, the elastase used in the present invention is porcine elastase (from the porcine pancreas), for example, E0127 (Sigma-Aldrich).

“Non-denatured type II collagen” means a type II collagen monomer that maintains a triple helix structure and/or a structure formed by the assembly of such monomers.

The term “collagen product” or “non-denatured type II collagen product” refers herein to any product containing type II collagen which has a triple helix structure. The “product” of the present invention refers to collagen-containing extract or powder (e.g., animal cartilage powder) obtained by processing collagen-containing raw material, as well as finished products made from the extract or powder by other processing step, e.g., drug, pharmaceutical, medical aesthetic injectable, etc., made by mixing the extract or powder with a pharmaceutically acceptable carrier, or collagen-containing food, health product, cosmetic, or other industrial product made by adding the extract or powder to food, health product, cosmetic, or other industrial product. In some embodiments, the collagen-containing raw material is known to those skilled in the art, such as various animal cartilage (e.g., bovine cartilage, chicken cartilage, pig snout bone, shark skull), fish bone, fish skin, etc., and the process for extracting collagen from raw material is also known to those skilled in the field.

The term “cartilage material” or “cartilage” is used herein to refer to any animal cartilage containing non-denatured type II collagen, such as chicken cartilage, bovine cartilage, etc.

The principle of the invention is as follows:

The water-soluble protein impurity (usually glycoproteins) and hydrolyzed collagen are first removed with guanidine hydrochloride, while non-denatured collagen and open-chain collagen are retained in the precipitate. The precipitate is suspended and sonicated under acidic condition to promote collagen swelling. Pepsin is added to enzymatically break down the denatured collagen, while the telopeptide of non-denatured collagen is removed and the triple helix region of collagen is freed. For pepsin-resistant type II collagen, elastase is added to further break the telopeptide, as well as to break the covalent bonds between non-denatured monomeric collagen molecules and between collagen and elastin, releasing water-soluble monomeric collagen molecules to accommodate different quantitative assays.

Ultrasonication shortens the time of pepsin digestion. Elastase digestion greatly increases the extraction rate of type II collagen. Based on the difference of advanced structure of different types of collagens, the three-stranded helical structure, i.e., non-denatured property, was confirmed by combining SDS-PAGE, circular dichroism, and other detections. The non-denatured type II collagen solution obtained by the method of the present invention may be further classified or subject to subsequent quantitative method. Liquid-mass spectrometry may be used for qualitative or quantitative analysis based on the amino acid sequence of the different types of collagens. ELISA assay was used to determine the properties of natural type II collagen and to quantify it in examples of the invention.

After obtaining a sample of non-denatured collagen in a solubilized state by the method of the present invention, qualitative and/or quantitative analysis of collagen is well known to those skilled in the art, for example, qualitative analysis by SDS-PAGE, quantitative analysis by ELISA or and/or high-performance liquid chromatography/mass spectrometry, etc.

The present invention provides a method for separating non-denatured type II collagen from a complex environment (including various collagen products and cartilage raw materials) and presenting it in a solubilized state. The method of the present invention are simple and easy to operate with high accuracy, which is very effective and practical. It is likely that industrially produced or prepared collagen or collagen products are partially or completely denatured during the production or preparation process, and many of the beneficial effects of the products or collagen can be provided only by non-denatured collagen. The use of the method of the present invention allows for accurate determination of the amount of non-denatured collagen in the product, facilitating quality control, quality assurance, and further optimization of production and preparation steps. In addition, the method can also be used to determine the content of non-denatured type II collagen in cartilage raw material, as well as for the extraction of non-denatured collagen from cartilage raw materials.

### BRIEF DESCRIPTION OF DRAWINGS

The present invention will be better understood by the following detailed description and drawings, wherein similar elements are numbered in a similar manner, wherein:

FIG. 1 SDS-PAGE electrophoresis of non-denatured type II collagen obtained from non-denatured type II collagen-containing chicken cartilage powder.

FIG. 2 Circular dichroism of non-denatured type II collagen extracted from chicken cartilage powder containing non-denatured type II collagen

### EMBODIMENTS

To have a clearer description of the technical features and effects of the present invention, the following examples are used to illustrate specific embodiments of the invention, which are only some but not all of the examples of the present invention. They are not intended to limit the scope of the present invention. Any equivalent or variation made within the spirit of the invention, e.g., combination, division or repetition of features, are all included in the protection scope of the invention.

Type II collagen is cartilage collagen, which accounts for more than 95% of the total collagen content in cartilage. Example 1 and Example 2 were using chicken cartilage powder and chicken cartilage for example, respectively, because their collagen composition is relatively simple and consists essentially of a small amount of denatured type II collagen and a large amount of non-denatured type II collagen. Example 1 compared this method with the HYP method for quantification to illustrate this method's accuracy for quantifying non-denatured type II collagen. Example 2 compared this method with two quantitative methods commonly used in the literature, i.e., acid extraction and pepsin extraction. Example 3 used a pressed candy containing several types of natural non-denatured collagen to illustrate the specificity of this method for non-denatured type II collagen.

### Example 1 Determination of Non-Denatured Type II Collagen Content in Chicken Cartilage Powder Containing Non-Denatured Type II Collagen

Chicken cartilage powder containing non-denatured type II collagen is an industrial product prepared from chicken cartilage by degreasing, decalcifying, partial purification, and drying processes etc., which maintains the triple helix structure of type II collagen as much as possible, while part of type II collagen is inevitably denatured or even degraded due to heat and other factors during this series of processing.

### (1) Pre-Treatment

Add 8 mL of 0.1 mol/L Tris-HCl buffer (containing 25 mmol/L NEM) containing 4 mol/L MgCl₂ into 0.3 g of chicken cartilage powder containing non-denatured type II collagen which was ground to 100-200 mesh, mix well, shake overnight at 4° C. (at least 16 h), and centrifuge at 9000 rpm for 10 min. Add 10 mL of 0.1 mol/L Tris-HCl buffer (containing 25 mmol/L NEM) containing 4 mol/L guanidine hydrochloride into the precipitate, shake overnight at 4° C. (at least 16 h), and centrifuge at 9000 rpm for 10 min again. The precipitate was resuspended with 10 mL of pre-cooled deionized water, shaken for 1 h. and centrifuged at 9000 rpm for 10 min; repeated once.

Add 10 mL of 0.05 mol/L acetic acid solution to the precipitate obtained from the above centrifugation and mix well. Adjust the pH to 2.5 with formic acid, sonicate at 20° C. for 10 min, centrifuge at 9000 rpm for 10 min, collect the supernatant, suspend the precipitate again with 10 mL of acetic acid, sonicate at 20° C. for 10 min, combine with the supernatant obtained from the previous centrifugation, and stir well. The pH of the combined solution was adjusted to 2.5 with formic acid, then 20 mg of pepsin was added to the combined solution, and the solution was digested for at least 16 h at 25° C. 2 mL of 10×TBS buffer was added to the resulting digest solution, and the pH was adjusted to 8.0 with 2 mol/L NaOH, and the total volume of the solution (V₁) was recorded. Take 1 mL of the alkaline-adjusted pepsin digest (V₃), add 9 mL of 1×TBS solution, then add 1 mg of elastase (Sigma-Aldrich), digest at 4° C. for 16 h, centrifuge, and record the volume of supernatant (V₂).

### (2) Characterization

Since the final enzymatic digest obtained by the present invention contains, in addition to non-denatured type II collagen, hydrolyzed collagen contained in the sample to be tested as well as collagen released by hydrolysis of open-chain type II collagen during the pretreatment process, the non-denatured type II collagen in the final enzymatic digest was separated by salinization and characterized.

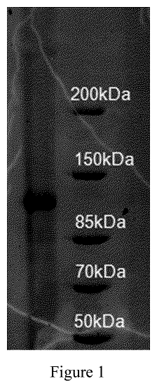

To the supernatant obtained after centrifugation of elastase digest according to the steps of Example 1 (1), NaCl at a final concentration of 2 mol/L was added for salinization, and the precipitate was dialyzed with 0.05 mol/L PBS (pH 7.4) for 24 h, and the dialysate was changed every 4 h. The dialyzed solution was mixed with 4× electrophoresis loading buffer and subjected to SDS-PAGE electrophoresis analysis with a 5% concentration of concentration gel and an 8% concentration of separation gel. As shown in FIG. 1, there was an obvious band in the sample profile with a molecular weight of about 113 kDa analyzed by gel chromatography imaging software, which was consistent with the report in the literature that chicken type II collagen consists of three identical polypeptide chains (al chain) with a molecular weight of about 110 kDa, and a fainter band at >200 kDa, which should be the dimeric β chain of α1.

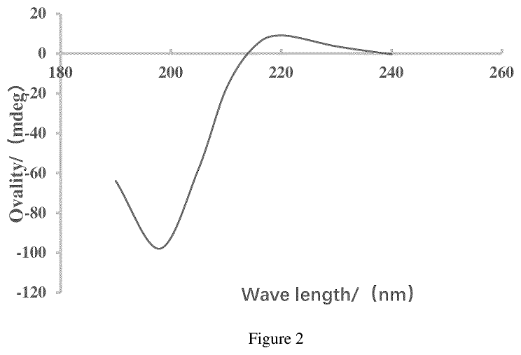

The circular dichroism of the protein solution can provide information on the secondary structure of the protein, and the circular dichroism of the non-denatured type II collagen after salinization and dialysis was shown in FIG. 2. There was a strong negative absorption peak and a weak positive absorption peak at 200 nm and 230 nm, respectively, which are typical features of circular dichroism of peptide chain in the L-polyproline configuration, indicating that the final enzymatic digest maintained the three-stranded helical structure of natural collagen.

The SDS-PAGE electrophoresis and circular dichroism analysis of the salinization product showed that the extract was non-denatured type II collagen retaining the triple-helical structure.

### (3) Detection

The final enzymatic supernatant obtained according to the procedure of Example 1 (1) was diluted at 3 concentration levels with PBS buffer (pH 7.4) according to the instructions of the chicken non-denatured type II collagen ELISA kit (e.g. JL45916), and each concentration level was replicated and tested, and the results were the average of 6 sets of values. The content of non-denatured type II collagen in the sample (X, mg/g) was calculated according to the following equation \textcircled{1}.

X=(C×N×V₂×(V₁/V₃))/(m×10⁶) \textcircled{1}
where X is the content of non-denatured type II collagen in the sample of chicken cartilage powder to be tested which contains non-denatured type II collagen, in mg/g; C is the concentration of non-denatured type II collagen in the sample solution on the kit, in ng/mL; N is the dilution ratio of the final enzymatic supernatant diluted to the sample solution on the kit in Example 1 (1); V₁ is the volume of the solution after alkaline-adjustment of the pepsin enzymatic digest in Example 1 (1), in mL; V₃ is the volume of the solution removed from the solution after alkaline-adjustment of the pepsin enzymatic digest in Example 1 (1); V₂ is the volume of the final enzymatic supernatant in Example 1 (1), in mL; m is the weight of the chicken cartilage powder to be tested in g; 10⁶ is the unit conversion factor.

### (4) Repeatability

The content of non-denatured type II collagen in chicken cartilage powder sample containing non-denatured type II collagen was tested for six times according to the steps of Example 1 (1)-(3), and the results were shown in Table 1.

TABLE 1
Content of non-denatured type II collagen in chicken cartilage
powder containing non-denatured type II collagen

| | | | | | | | | RSD |
|---|---|---|---|---|---|---|---|---|
| Parallelism | 1 | 2 | 3 | 4 | 5 | 6 | [symbol] | % |
| Non-denatured | 243 | 262 | 258 | 259 | 261 | 247 | 254 | 4.0 |
| type II collagen | | | | | | | | |
| content mg/g | | | | | | | | |

### (5) Intermediate Precision

The same batch of chicken cartilage powder containing non-denatured type II collagen was sampled and tested by the same operator according to the pre-treatment method of Example 1 (1) and the testing steps of Example 1 (3) at six different times on Feb. 19, 2020, March 9 and 23, 2020, April 13 and 22, 2020 and May 13, 2020, respectively, and the results were shown in Table 2.

The same batch of chicken cartilage powder was sampled and tested by three experimenters in our laboratory according to the pre-treatment method of Example 1 (1) and the testing steps of Example 1 (3), respectively, and the results were shown in Table 2.

TABLE 2
Intermediate precision of non-denatured type II
collagen content in chicken cartilage powder

| Experiment date | 2.19 | 3.9 | 3.23 | 4.13 | 4.22 | 5.13 |
|---|---|---|---|---|---|---|
| Non-denatured | 258 | 249 | 274 | 261 | 277 | 257 |
| type II collagen | | | | | | |
| content mg/g | | | | | | |

Average = 260,
RSD = 4.9%

| Experimenter | A | B | C |
|---|---|---|---|
| Non-denatured | 266 | 275 | 253 |
| type II collagen | | | |
| content mg/g | | | |

[symbol]

= 265 mg/g,
RSD = 4.2%

As can be seen from Table 2, the RSDs of the experimental results were all less than 5%, indicating that the experimental method has a high stability in the results when tested at different times or operated by different persons, which shows the high intermediate precision of the method.

### (6) Determination of Non-Denatured Type II Collagen Content in Chicken Cartilage Powder by HYP Method

The non-denatured type II collagen has an intact triple helix structure and cannot be digested by trypsin. Following the experimental procedure of the patent (CN108659117B “A method for quantitative detection of the content of the triple helix structure of collagen”), the HYP content in the remaining chicken cartilage powder of the same batch of Example 1 (1) was determined after trypsin digestion. The conversion factor of HYP and type II collagen was 7.4 (NY/T 3608-2020 Method for the determination of bone collagen content in livestock and poultry, Spectrophotometric method). The contents of non-denatured type II collagen in chicken cartilage powder measured by the HYP method were shown in Table 3. The results obtained by the present method were close to them.

TABLE 3
Determination of non-denatured type II collagen
content in chicken cartilage powder by HYP method

| | | | | | | | | RSD |
|---|---|---|---|---|---|---|---|---|
| Parallelism | 1 | 2 | 3 | 4 | 5 | 6 | [symbol] | % |
| Non-denatured | 252 | 260 | 258 | 269 | 241 | 270 | 259 | 3.9 |
| type II collagen | | | | | | | | |
| content mg/g | | | | | | | | |

### Example 2 Determination of Non-Denatured Type II Collagen Content in Chicken Breast Cartilage

### (1) Pre-Treatment

Take 1 g of chicken breast cartilage with impurities such as muscle and fascia removed and cut up. Add 15 mL of pre-cooled deionized water, homogenate and discard the supernatant. Add 15 mL of pre-cooled deionized water to the precipitate again, homogenate and repeat twice. Add 20 mL of chloroform-methanol-water (1:2:0.8) solution to the washed precipitate, mix and shake several times, remove the chloroform layer, centrifuge the slurry layer at 4000 rpm for 10 min, and wash the precipitate for 3 times with water. 20 mL of 0.1 mol/L Tris-HCl buffer (containing 25 mmol/L NEM) with 4 mol/L guanidine hydrochloride was added to the precipitate and mixed, shaken overnight at 4° C. (at least 16 h), and centrifuged at 9000 rpm for 10 min. The precipitate was resuspended with 20 mL of pre-cooled 0.1 mol/L Tris-HCl buffer (containing 25 mmol/L NEM), gently shaken for 1 h, and centrifuged at 9000 rpm for 10 min; repeat twice.

Add 10 mL of 0.05 mol/L acetic acid solution to the precipitate obtained from the above centrifugation and mix well. Adjust the pH to 3.0 with formic acid, sonicate at 25° C. for 10 min (pulse frequency 5 s/5 s), centrifuge at 9000 rpm for 10 min, collect the supernatant, suspend the precipitate with 10 mL of 0.05 mol/L acetic acid, sonicate at 25° C. for 10 min, and combine with the supernatant from the previous centrifugation. The pH of the combined solution was adjusted to 2.8 with formic acid, and 20 mg of pepsin (Sigma-Aldrich) was added to the combined solution for enzymatic digestion at 30° C. for 6 h. 2 mL of 10×TBS buffer was added to the resulting enzymatic digest, and the pH was adjusted to 8.1 with 2 mol/L NaOH, and the total volume of the enzymatic digest after alkaline-adjustment (V₁) was recorded. Add 1 mL of the aforementioned solution (V₃) to 10 mL of 1×TBS solution, add 1 mg of elastase (Sigma-Aldrich) for enzymatic digestion at 4° C. for 16 h, and centrifuge at 1000 rpm for 20 min. Store the supernatant (V₂) at 4° C., and dilute the final enzymatic digest with PBS (pH 7.4) buffer to the desired concentration before testing.

The reagents involved in the examples are all analytically pure. 10×TBS solution is a 1 mol/L Tris solution containing 0.4 mol/L NaCl and 10 mmol/L CaCl₂). 1×TBS solution could be obtained by diluting the 10×TBS solution for 10 times.

### (2) Results

The detection and result calculation of non-denatured type II collagen in chicken cartilage powder containing non-denatured type II collagen were performed according to Example 1 (3).

### A. Repeatability

Commercially available chicken breast cartilage was processed to remove impurities such as muscle and fascia, then cut up and mixed well. Six parallel samples were tested and the content of non-denatured type II collagen was determined following the above pre-treatment method. The results were shown in Table 4.

TABLE 4
Content of non-denatured type II collagen in commercially
available chicken breast cartilage

| Parallelism | 1 | 2 | 3 | 4 | 5 | 6 |
|---|---|---|---|---|---|---|
| Non-denatured | 30.9 | 32.8 | 29.4 | 33.0 | 34.1 | 29.7 |
| type II collagen | | | | | | |
| content mg/g | | | | | | |

[symbol]

= 31.6 mg/g,
RSD = 6.1%

### B. Intermediate Precision

The same batch of chicken breast cartilage was tested by the same operator according to the method of the present invention at six different times on Mar. 24, 2020, April 3, 19, 2020, Apr. 26, 2020, May 13, 26, 2020, and all the other operational steps were the same as in Example 1, and the results were shown in Table 5.

The same batch of chicken breast cartilage was tested by three experimenters according to the method of the present invention, and all the other operational steps were the same as in Example 1, and the results were shown in Table 5.

TABLE 5
Intermediate precision of non-denatured type II
collagen content in chicken breast cartilage

| Experiment date | 3.24 | 4.3 | 4.19 | 4.26 | 5.13 | 5.26 |
|---|---|---|---|---|---|---|
| Non-denatured | 30.6 | 31.7 | 32.2 | 32.8 | 33.3 | 31.5 |
| type II collagen | | | | | | |
| content mg/g | | | | | | |

[symbol]

= 32.0 mg/g,
RSD = 3.1%

| Experimenter | A | B | C |
|---|---|---|---|
| Non-denatured | 31.4 | 33.9 | 32.4 |
| type II collagen | | | |
| content mg/g | | | |

[symbol]

= 32.6 mg/g,
RSD = 3.9%

As can be seen from Table 5, the RSDs of the experimental results were all less than 5%, indicating that the experimental method has a high stability in the results when tested at different times or operated by different persons, indicating the high intermediate precision of the method.

### Comparative Example 1 Acid Extraction of Non-Denatured Type II Collagen in Chicken Breast Cartilage

The method of Sajithlal et al. (Effect of Curcumin on the Advanced Glycation and Cross-linking of Collagen in Diabetic Rats) was used, with modifications, for acid extraction of non-denatured type II collagen from chicken breast cartilage. Steps are as follows:

The whole operation was performed at 4° C. 1 g of chicken breast cartilage with muscle, fascia, and other impurities removed was cut up, and washed thoroughly in PBS (pH 7.4) containing EDTA, PMSF, benzamidine HCl, and thioacetamide (all 1 mmol/L) protease inhibitor. Add 50 mL of 0.05 mol/L acetic acid and stir for 24 h. Homogenize the mixture and continue to stir for 24 h. Centrifuge at 1000 rpm for 60 min. The supernatant is the acid-extracted collagen.

The same batch of chicken breast cartilage as in Example 2 was taken and the acid extraction was repeated for six times. The results were shown below:

TABLE 6
Content of acid-extracted non-denatured type
II collagen in chicken breast cartilage

| Experiment | 1 | 2 | 3 | 4 | 5 | 6 |
|---|---|---|---|---|---|---|
| Content mg/g | 2.31 | 2.56 | 2.47 | 3.13 | 2.86 | 2.63 |

[symbol]

= 2.63,
RSD = 10.1%

### Comparative Example 2 Pepsin Extraction of Non-Denatured Type II Collagen in Chicken Breast Cartilage

According to Kochakian et al., (Chronic Dosing with Aminoguanidine and Novel Advanced Glycosylation End Product-Formation Inhibitors Ameliorates Cross-Linking of Tail Tendon Collagen in STZ-Induced Diabetic Rats) and Oturai et al. (Effects of Advanced Glycation End-Product Inhibition and Cross-Link Breakage in Diabetic Rats), pepsin extraction of non-denatured type II collagen from chicken breast cartilage was carried out as follows:

Take 1 g of chicken breast cartilage with impurities such as muscle and fascia removed and cut up. Add 50 mL of pepsin (1 mg/mL) acetic acid (1 mol/L) solution, stir at 4° C. for 48 h, and centrifuge at 9000 rpm for 20 min. The supernatant is pepsin-extracted collagen.

The pepsin extraction was carried out by taking the same batch of chicken breast cartilage as in Example 2, and the results of six replicates were as follows:

TABLE 7
Content of pepsin-extracted non-denatured type
II collagen in chicken breast cartilage

| Experiment | 1 | 2 | 3 | 4 | 5 | 6 |
|---|---|---|---|---|---|---|
| Content mg/g | 12.7 | 13.3 | 14.8 | 14.0 | 14.1 | 13.9 |

[symbol]

= 13.8,
RSD = 4.8%

By comparing Table 1, Table 6, and Table 7, it can be found that the results obtained by the method provided by the present invention are much greater than those of the commonly used acid extraction method and pepsin extraction method, and the RSD values are also low.

### Example 3 Determination of Non-Denatured Type II Collagen Content in Pressed Candy Containing Non-Denatured Type II Collagen

The non-denatured type II collagen-containing pressed candy was made from bovine bone collagen powder (type I and III collagen), non-denatured type II collagen-containing chicken cartilage powder (the same batch as Example 1), hydrolyzed type II collagen powder, and other excipients upon coloring, flavoring and pressing.

### (1) Pre-Treatment

1 g of non-denatured type II collagen-containing pressed candy which was milled to 100-200 mesh was added with 8 mL of 0.1 mol/L Tris-HCl buffer (containing 25 mmol/L NEM) with 4 mol/L MgCl₂, mixed, and shaken overnight at 4° C. (at least 16 h), centrifuged at 9000 rpm for 10 min, and the precipitate was suspended with 10 mL of 0.1 mol/L Tris-HCl buffer with 4 mol/L guanidine hydrochloride (containing 25 mmol/L NEM) and shaken overnight at 4° C. (at least 16 h), centrifuged at 9000 rpm for 10 min again. The precipitate was resuspended with 10 mL of pre-cooled deionized water, shaken for 1 h, and centrifuged at 9000 rpm for 10 min; repeated once.

Add 10 mL of 0.05 mol/L acetic acid solution to the precipitate resulting from the above centrifugation and mix well. Adjust the pH to 2.8 with formic acid, sonicate at 25° C. for 15 min, centrifuge at 9000 rpm for 10 min, collect the supernatant, suspend the precipitate with 10 mL of acetic acid, sonicate at 25° C. for 15 min, and combine with the supernatant from the previous centrifugation. The pH of the combined solution was adjusted to 2.8 with formic acid and 20 mg of pepsin was added to the combined solution for enzymatic digestion for at least 16 h at 30° C. 2 mL of 10×TBS buffer was added to the enzymatic digest, and the pH was adjusted to 8.1 with 2 mol/L NaOH, and the total volume of the suspension (V₁) was recorded. Take 1 mL of the suspension, add 9 mL of 1×TBS solution, then add 1 mg of elastase (Sigma-Aldrich) and digest for 16 h at 4° C. Dilute the final digest solution with PBS (pH 7.4) buffer to the desired concentration range and store for subsequent testing.

### (2) Assay

The non-denatured type II collagen of the above-pretreated pressed candy was tested and the results were calculated according to the method of Example 1 (3). The amount of chicken cartilage powder containing non-denatured type II collagen added to the pressed candy was 6%. The content of non-denatured type II collagen in the chicken cartilage powder was 25.4% which can be seen from the result of Example 1, so the theoretical content of non-denatured type II collagen in the pressed candy was 15.2 mg/g with a relative deviation of 1.97%. The actual results of the method of the invention were shown in Table 8, from which it can be seen that the relative deviation between the measured value and the theoretical value complies with the requirements of GB/T 27404-2008 Laboratory Quality Management Control Specification for Physical and Chemical Testing of Food.

The HYP method described in the aforementioned patent CN108659117B was used to test the same batch of pressed candy, and the results were shown in Table 8. The results showed that for products containing multiple types of non-denatured collagen, the HYP method similar to that described in patent CN108659117B is not able to quantify the active ingredient, i.e. non-denatured type II collagen.

TABLE 8
Content of non-denatured type II collagen in pressed candy
containing non-denatured type II collagen (mg/g)

| Parallelism | 1 | 2 | 3 | 4 | 5 | 6 | [symbol] | RSD (%) | RD (%) |
|---|---|---|---|---|---|---|---|---|---|
| Method of the present | 14.9 | 15.6 | 14.9 | 16.6 | 15.3 | 15.7 | 15.5 | 2.7 | 1.97 |
| invention | | | | | | | | | |
| HYP method | 22.4 | 22.9 | 23.7 | 21.0 | 23.2 | 21.6 | 22.5 | 4.2 | 48.0 |

1. A pre-treatment method for detection of non-denatured type II collagen in a collagen product or cartilage raw material, comprising:
(1) adding a buffer solution containing a neutral salt or guanidine hydrochloride to a sample of the collagen product or cartilage raw material to be tested, centrifuging, and obtaining a precipitate;
(2) resuspending the precipitate obtained in (1) with an acid, accelerating the swelling by ultrasonication, and centrifuging to obtain a supernatant;
(3) adding pepsin to the supernatant obtained in (2) for enzymatic digestion; and
(4) adding elastase to the enzymatic digest obtained in (3) for enzymatic digestion, centrifuging, and retaining the supernatant as the solution to be tested.

2. A method for detecting non-denatured type II collagen in a collagen product or cartilage raw material, comprising:
(1) adding a buffer solution containing a neutral salt or guanidine hydrochloride to a sample of the collagen product or cartilage raw material to be tested, centrifuging, and obtaining a precipitate;
(2) resuspending the precipitate obtained in (1) with an acid, accelerating the swelling by ultrasonication, and centrifuging to obtain a supernatant;
(3) adding pepsin to the supernatant obtained in (2) for enzymatic digestion;
(4) adding elastase to the enzymatic digest obtained in (3) for enzymatic digestion, centrifuging, and retaining the supernatant as the solution to be tested; and
(5) performing qualitative and/or quantitative analysis of collagen on the solution to be tested obtained in (4).

3. The method of claim 1, wherein the concentration of guanidine hydrochloride in the buffer solution containing a neutral salt or guanidine hydrochloride in (1) is in the range of 1-6 mol/L.

4. The method of claim 1, wherein the buffer solution containing a neutral salt or guanidine hydrochloride in (1) is prepared by a method comprising dissolving the neutral salt or guanidine hydrochloride in 0.1 mol/L Tris-HCl buffer and stirring well.

5. The method of claim 1, wherein the power of the ultrasonication in (2) is 100-500 W.

6. The method of claim 1, wherein the duration of the ultrasonication in (2) is 1-15 min.

7. The method of claim 1, wherein the concentration of said pepsin solution in (3) is 0.1-5 mg/mL.

8. The method of claim 1, wherein the duration of the enzymatic digestion in (3) is 16-72 h and the temperature of the enzymatic digestion is 4° C.-30° C.

9. The method of claim 1, wherein said elastase solution in (4) has a concentration of 0.01-1 mg/mL.

10. The method of claim 1, wherein the duration of the enzymatic digestion in (4) is 10-30 h and the temperature of the enzymatic digestion is 2° C.-10° C.

11. The method of claim 1, wherein (2) further comprises adjusting the pH to 2.0-3.0 after resuspending precipitate.

12. The method of claim 1, wherein (4) further comprises adjusting the pH of the enzymatic digest obtained in (3) to 7.5-9.0 before the addition of elastase.

13. The method of claim 1, wherein the concentration of guanidine hydrochloride in the buffer solution of guanidine hydrochloride is 3-4 mol/L.

14. The method of claim 1, wherein the duration of the enzymatic digestion in (3) is 16-20 h.

15. The method of claim 1, wherein in (4), said elastase has a final concentration in the solution of 0.1 mg/mL.

16. The method of claim 1, wherein the duration of the enzymatic digestion in (4) is 16-22 h.

17. A kit for the detection of non-denatured type II collagen in a collagen product or cartilage raw material, wherein said kit comprises a buffer solution containing a neutral salt or guanidine hydrochloride, a pepsin solution, and an elastase solution.

18. The kit of claim 17, wherein the concentration of guanidine hydrochloride in the buffer solution containing guanidine hydrochloride is between 1-6 mol/L, preferably 3-4 mol/L.

19. The kit of claim 17, wherein the buffer solution comprising a neutral salt or guanidine hydrochloride is prepared by a method comprising dissolving the neutral salt or guanidine hydrochloride in 0.1 mol/L Tris-HCl buffer and stirring well.

20. The kit of claim 17, wherein the concentration of pepsin in said pepsin solution is 0.1-5 mg/mL.

21. (canceled)
